(19) United States
(12) Patent Application Publication
(10) Pub. No.: US 2018/0217118 A1
(43) Pub. Date: Aug. 2, 2018

(54) SYSTEM AND METHOD FOR DETERMINING PRODUCT PERISHABILITY

(71) Applicant: Wal-Mart Stores, Inc., Bentonville, AR (US)

(72) Inventors: Michael L. Payne, Centerton, AR (US); Joshua R. Freeman, Bentonville, AR (US); Jacob A. Jones, Centerton, AR (US); Winston E. D. Spencer, Bentonville, AR (US)

(21) Appl. No.: 15/861,247

(22) Filed: Jan. 3, 2018

Related U.S. Application Data
(60) Provisional application No. 62/453,327, filed on Feb. 1, 2017.

(51) Int. Cl. G01N 33/02; G01K 1/02; A23L 3/36; G06Q 10/08

(52) U.S. Cl. CPC G01N 33/02; G06Q 10/087; A23L 3/36; G01K 1/022

(57) ABSTRACT

An average spoilage temperature for a product is obtained. The average spoilage temperature is the temperature when the product becomes not viable for customer use. In real-time, the current temperature of the retail store in which the product resides is measured. In real-time, a perishability time for the product is calculated using the average spoilage temperature of the product, and the current temperature of the retail store. In real-time, the location of the product and the time since the product has left a first shelf are tracked. The first shelf is a location where a shelf temperature is maintained to adequately preserve the product. In real-time, the perishability time is compared to the time since the product has left the first shelf. When the time since the product has left the first shelf exceeds the perishability time, an action is taken.

### CROSS-REFERENCE TO RELATED APPLICATION

This application claims the benefit of the following U.S. Provisional Application No. 62/453,327 filed Feb. 1, 2017, which is incorporated herein by reference in its entirety.

### TECHNICAL FIELD

This invention relates generally to tracking the perishability of products.

### BACKGROUND

Many products need to be maintained in a controlled environment. If the proper environmental conditions are not maintained, then the product becomes spoiled or otherwise becomes unusable. For example, frozen products need to be maintained in freezers or these products will spoil. Other products need to be cooled, or they will spoil. Still other products cannot fall below a certain temperature, or these products become unusable. For example, some stores selling pre-cooked items (e.g., pizza) maintain the items in a heated environment. If the product is removed from the heated environment and cools, customers (who wish to consume only warm items) will not purchase the product.

Products are moved from their controlled environments for a number of reasons. Most often, a customer wishes to purchase the item. However, sometimes the customer removes the item and for some reason decides not to purchase the item. For instance, a customer may remove a frozen item, proceed to the checkout, decide not to purchase the item, then remove the item and place it on an open shelf (that is not environmentally controlled) near the checkout. In such situations, the item may completely defrost and become spoiled.

### BRIEF DESCRIPTION OF THE DRAWINGS

Disclosed herein are embodiments of systems, apparatuses and methods pertaining to tracking the perishability of products. This description includes drawings, wherein:

FIG. 1 is a block diagram for determining the perishability condition of a product in accordance with some embodiments;

FIG. 2 is a block diagram showing movement of a product through a store in accordance with some embodiments;

FIG. 3 is a flowchart of one example of an approach for determining the perishability condition of a product in accordance with some embodiments.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to various embodiments, systems, apparatuses and methods are provided herein that calculate the perishability time of a product in a retail store. A product normally resides at a shelf (or other storage unit) where environmental conditions are maintained to preserve the product. When the product leaves the shelf (or other storage unit) of a retail store, the running time since the product has left the shelf is continuously measured, and the perishability time is dynamically compared to this running time. When the running time exceeds the perishability time, the product is assumed to have perished or have become otherwise unusable. Various actions can then be taken to preserve the product (before it spoils), or dispose of the product (after it is assumed to have spoiled).

Advantageously, the location of products can be tracked, and products can be returned to their proper location to be preserved. Good products that might have been erroneously thrown away are preserved thereby reducing costs. For example, store employees no longer have to automatically throw away frozen items that have left their shelves due to customers placing the items in other parts of the stores. The employees are able to find these items easily and place them back in their respective areas increasing the availability of items to customers. The amount of inventory due lost due to spoilage is reduced. Faster response times are achieved aiding in the prevention of unnecessary inventory shrinkage.

On the other hand, products that have spoiled are discarded. Advantageously, the sale of spoiled products to customers is prevented.

The system also has the ability to learn and adapt to changing circumstances. Additionally, the present approaches can be used to track products and predict/identify when an item is misplaced in the store. This information can be communicated to store employees to take corrective actions.

In some of these embodiments, a system includes a database, a temperature sensor, a location tracking sensor, and a control circuit. The database is configured to store an average spoilage temperature for a product. The average spoilage temperature is the temperature when the product becomes not viable for customer use.

The temperature sensor is configured to measure the temperature of the retail store in which the product resides. The location tracking sensor is configured to track the location of the product and, in real-time, to track the time since the product left a shelf (or other storage location). The shelf is a location where a shelf temperature (or other environmental conditions) is maintained to adequately preserve the product.

The control circuit is coupled to the database, the temperature sensor, and the location tracking sensor. The control circuit is configured to, in real-time, calculate a perishability time for the product using the average spoilage temperature of the product and the current temperature of the retail store.

The control circuit is further configured to compare the perishability time to the time since the product has left the first shelf. The control circuit is additionally configured to form and transmit a control signal to cause an action to occur when the time since the product has left the first shelf exceeds the perishability time.

In aspects, the control signal is an alert to a customer at a point-of-sale location (e.g., the checkout counter) in the retail store. In other examples, the control signal is an alert to a customer as the customer traverses through the retail store. In still other examples, the control signal is an alert to an employee of the store.

In some examples, the product is coupled to an RFID tag. In aspects, the location sensor senses the RFID tag and tracks the location of the RFID tag as the product moves through the store.

In other aspects, the average spoilage temperature is the temperature above which the item perishes. Alternatively, the average spoilage temperature is the temperature below which the product perishes.

In others of these embodiments, an average spoilage temperature for a product is obtained. The average spoilage temperature is the temperature when the product becomes not viable for customer use. In real-time, the current temperature of the retail store in which the product resides is measured. In real-time, a perishability time for the product is calculated using the average spoilage temperature of the product, and the current temperature of the retail store. In real-time, the location of the product and the time since the product has left a shelf (or other storage device) are tracked. The shelf is a location where a shelf temperature is maintained to adequately preserve the product. In real-time, the perishability time is compared to the time since the product has left the shelf. When the time since the product has left the first shelf exceeds the perishability time, an action is taken.

In some aspects, the action is alerting a customer at a point-of-sale location. In other examples, the action is alerting a customer as the customer traverses through the retail store. In still other examples the action is alerting an employee of the store. Other examples of actions are possible.

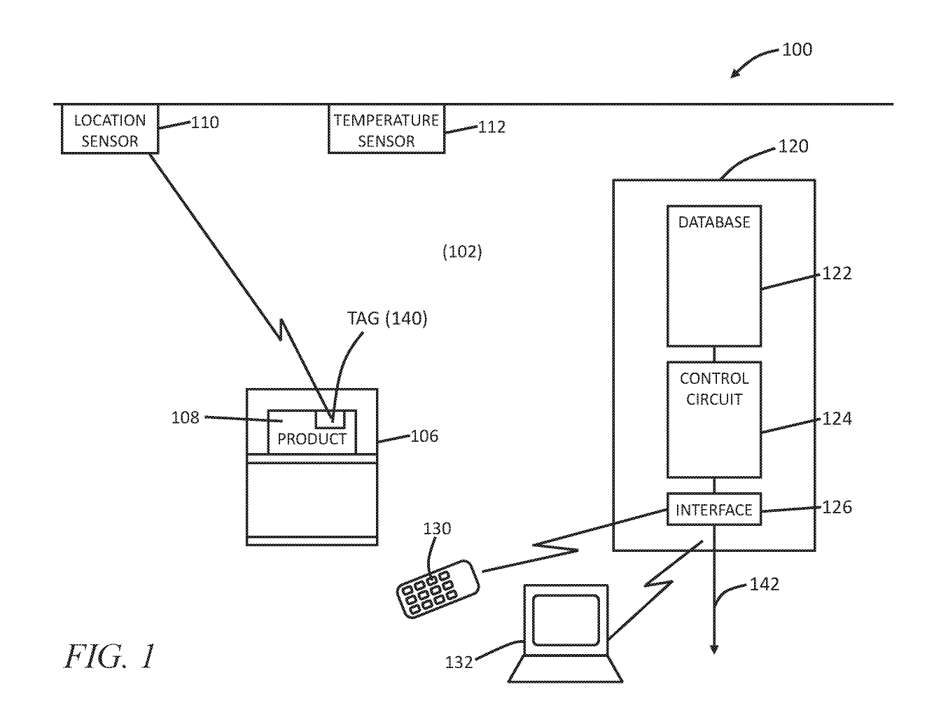

Referring now to FIG. 1, one example of a system for determining the spoilage condition of a product is described. The system 100 tracks products as the products leave a shelving unit (that is environmentally controlled) to ensure that the product does not spoil or become otherwise unusable. Many of the examples described herein utilize shelves or shelving units as storage units or devices. These shelves may be enclosed by various structures (e.g., glass doors). It will be appreciated that any environmentally controlled storage unit or device can also be utilized such as an open freezer (with no shelves), or a heated oven (e.g., storing heated products and with or without shelves).

The system 100 is deployed in a retail store 102. Within the retail store 102, a shelving or storage unit 106 holds a product 108. A location sensor 110 and a temperature sensor 112 are also deployed within the retail store 102. An apparatus 120 includes a database 122, a control circuit 124, and an interface 126. The interface 126 communicates with a smartphone 130 and a personal computer 132.

The retail store 102 may be any type of retail establishment that is open to the public for the purchase of products. The retail store 102 includes all areas, elements, and structures needed to present the products to customers for sale. For example, the retail store 102 includes display units to display the products, point-of-sale structures (e.g., cash registers and check-out areas) to allow the customer to purchase items, storage areas (e.g., back rooms) to store products before products are displayed, and receiving areas where products can be received from shippers. Other areas, elements, and structures are possible.

The shelving or storage unit 106 is configured to present products to customers for purchase. In examples, the unit 106 includes shelves where products are placed or held. Additionally, the shelving or storage unit 106 is environmentally controlled. For example, the shelving unit 106 may include a refrigeration unit that cools products and keeps the products at a predetermined temperature (or within a predetermined temperature range). In other examples, the shelving or storage unit 106 includes a heater unit, or warmer unit that heats a product to ensure that the product does not fall below a certain temperature or below a certain rage of temperatures.

In some examples, the shelving or storage unit 106 includes multiple shelves and is accessible by glass doors. The doors ensure that a predetermined temperature (or range of temperatures) are maintained in the unit, but also allow customers to easily view the product. For example, the shelving or storage unit 106 may be of the type typically found in grocery or discount stores having a glass door that is opened by a customer to access and retrieve a product.

In other examples, the shelving or storage unit 106 may be at least partially open (e.g., without doors). To take one specific example, the shelving or storage unit 106 may be an open freezer typically found in grocery or discount stores where a customer reaches downward to retrieve a product.

The product 108 is any product subject to spoilage. In aspects, the product may be a frozen product (e.g., frozen vegetables) or a cooled product (e.g., milk or eggs) that will spoil if the temperature of the product exceeds a predetermined temperature or range of temperatures. In other examples, the product may be a heated product (e.g., pizza) that will spoil or become otherwise unusable, if the product 108 falls below a predetermined temperature or range of temperatures.

The location sensor 110 tracks the product 108. In aspects, the location sensor 110 senses a tag 140 that is attached to the product 108. In some examples, the tag 140 is an RFID tag. The location sensor 110 communicates with the apparatus 120. The location sensor 110 is configured to track the location of the product and, in real-time, to track the time since the product left the shelf or storage unit 106.

In still other aspects, the tag 140 may also sense the temperature of the product 108 and transmit this information to the location sensor 110, which relays the sensed product temperature to the apparatus 120. The sensed product temperature may also be used in some examples to calculate the perishability time.

In yet other aspects, the sensed product temperature is used to verify assumptions about how the temperature of the product changes over time. This, in turn, provides for a more accurate calculation of perishability time. For instance, when the assumption is that the product's temperature will rise one degree per minute, but the tag 140 indicates the product is actually rising only 0.5 degrees per minute, then the perishability time can be adjusted accordingly.

The temperature sensor 112 is any type of sensing device that is configured to sense an air temperature. In this case the temperature sensor 112 senses the air temperature of the retail store 102. The temperature sensor 112 communicates with the apparatus 120. The temperature sensor is configured to measure the temperature of the retail store in which the product resides. In examples, the temperature sensor 112 is any type of temperature sensing device such as devices that include a thermometer. It also has the ability to transmit the temperature information over a wired connection or over a wireless connection to the control circuit.

As mentioned, the apparatus 120 includes the database 122, control circuit 124, and interface 126. The database 122 is configured to store an average spoilage temperature for the product 108. The average spoilage temperature is the temperature when the product becomes not viable for customer use.

The interface 126 communicates with a smartphone 130 and a personal computer 132. The interface 126 may be any combination of electronic hardware or computer software elements. Additionally, any other type of electronics device may be coupled to the interface 126.

The term “control circuit” refers broadly to any microcontroller, computer, or processor-based device with processor, memory, and programmable input/output peripherals, which is generally designed to govern the operation of other components and devices. It is further understood to include common accompanying accessory devices, including memory, transceivers for communication with other components and devices, etc. These architectural options are well known and understood in the art and require no further description here. The control circuit 124 may be configured (for example, by using corresponding programming stored in a memory as will be well understood by those skilled in the art) to carry out one or more of the steps, actions, and/or functions described herein.

The control circuit 124 is configured to, in real-time, calculate a perishability time for the product 108 using the average spoilage temperature of the product and the current temperature of the retail store 102. In aspects, a linear relationship can be used to calculate the time. For example, when the retail store is 70 degrees, the product may increase in temperature one-half (0.5) a degree per minute. If the initial product temperature is 32 degrees and spoilage occurs at 55 degrees, then the perishability time is 46 minutes [perishability time=0.5(55-32)].

In this example, the average spoilage temperature is a temperature above which the item perishes. In other aspects, the average spoilage temperature is a temperature below which the product perishes.

The control circuit 124 is configured to compare the perishability time to the time since the product 108 has left the shelving or storage unit 106.

The location sensor 110 may periodically (or continuously) send signals to the control circuit 124 with the coordinates of the product 108. The control circuit 124 may compare the coordinates to coordinates of the shelving or storage unit 106. When the coordinates change (or change by more than a predetermined amount) indicating that the product is outside the shelving or storage unit 106, the control circuit 124 may start a timer (e.g., either a hardware or software timer). The control circuit 124 uses this elapsed time recorded by the timer in its calculations.

The control circuit 124 is configured to form and transmit a control signal 142 to cause an action to occur when the time since the product has left the shelving or storage unit 106 exceeds the perishability time. In aspects, the control signal 142 is an alert to a customer at a point-of-sale location in the retail store 102. In other examples, the control signal 142 is an alert to a customer as the customer traverses through the retail store 102. In still other examples, the control signal 142 is an alert to an employee of the store. Other examples are possible.

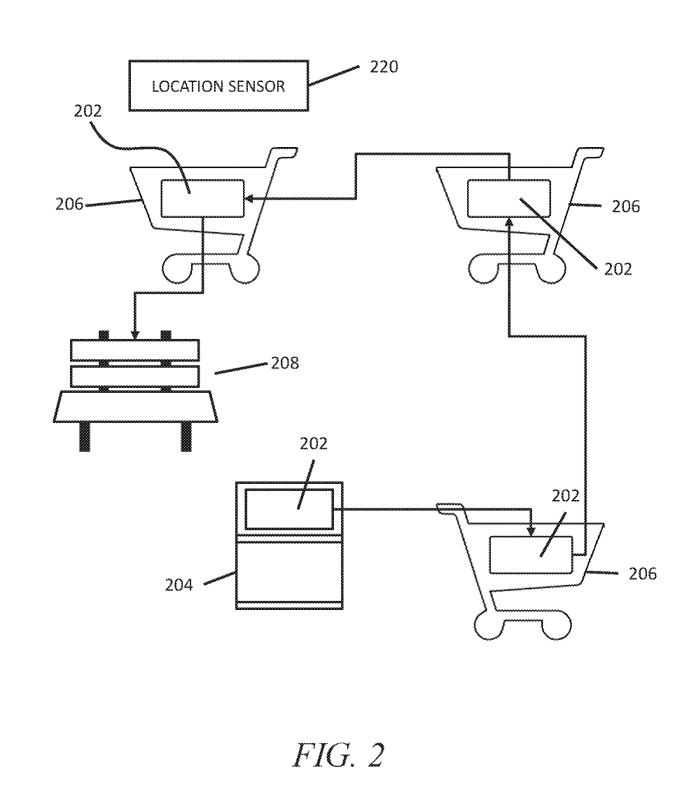

Referring now to FIG. 2, one example of deploying the present approaches in a retail store is described. A customer removes a product 202 from a shelving or storage unit 204 and places the product 202 in a shopping cart 206. The storage unit 204 is configured so as to preserve the product.

The cart 206 is pushed by the customer through the retail store. Eventually, the customer takes the product from the cart 206 and places the product on a second shelf 208. The second shelf 208 is not environmentally controlled so as to prevent the spoilage of the product.

A location sensor 220 tracks the product 202 as the product 202 moves through the retail store. In aspects, the product 202 includes, or has attached to it an RFID tag (or other type of tracking device). The location sensor 220 tracks the RFID tag as the product 202 moves along a path 222, eventually halting its movement at the second shelf 208. Various types of tracking approaches can be used to track the tag (and hence the product) as the product moves through the store.

The average spoilage temperature for the product 202 is also obtained and may be stored in a database. The database may be any type of memory device that stores information. The average spoilage temperature is the temperature when the product 202 becomes not viable for customer use. In real-time, the current temperature of the retail store in which the product 202 resides is also measured, for example, by a temperature sensor such as a thermometer.

In real-time, a perishability time for the product 202 is calculated using the average spoilage temperature of the product, and the current temperature of the retail store. In real-time, the location of the product 202 and the time since the product 202 has left the shelving or storage unit 204 are tracked. In real-time, the perishability time is compared to the time since the product has left the shelving or storage unit 204.

When the time since the product 202 has left the shelving or storage unit 204 exceeds the perishability time, an action is taken. The action may be an alert to a store employee to find the product and return the product 202 to a location where the product can be preserved (e.g., the shelving or storage unit 204). The employee may use information supplied (e.g., an exact physical location) to locate where the product is disposed.

In other examples, the tracking of the product 202 includes tracking the relative movements of the product 202. Thus, the system may determine that the product 202 is moving through the store, and may track the length of time that movement has halted. When the movement is halted for a predetermined amount of time and the product 202 is not at a location where movement is expected to be halted, then a determination may be made that the product 202 was dropped (or otherwise discarded) by the customer and the product 202 can be retrieved.

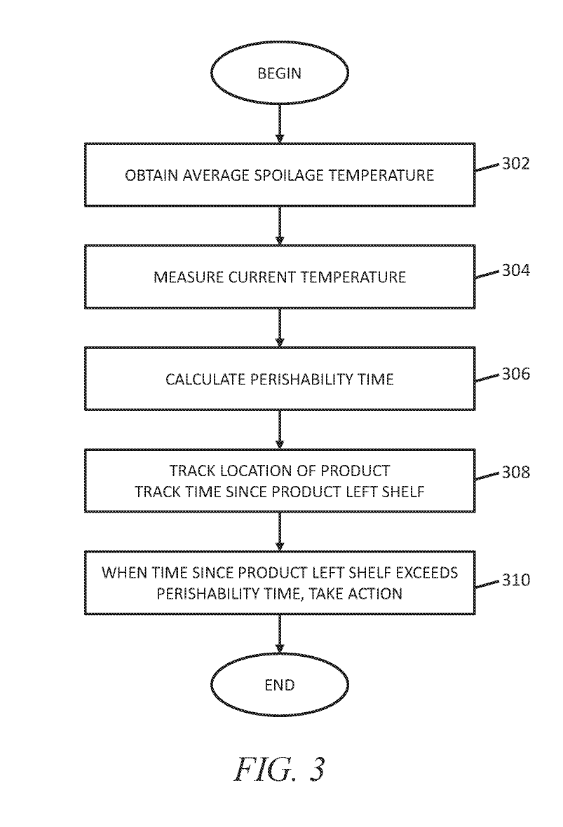

Referring now to FIG. 3, one example of an approach for determining the spoilage condition of a product is described. At step 302, an average spoilage temperature for a product is obtained. The average spoilage temperature is the temperature when the product becomes not viable for customer use. In some examples, the average spoilage temperature is a temperature above which the item spoils. In yet other examples, the average spoilage temperature is a temperature below which the product spoils.

The average spoilage temperature may be obtained in a variety of different ways. For example, a user may enter the temperature using some type of interface (e.g., a graphical user interface or a keypad). The spoilage temperatures for various products can be stored at a database in any suitable data structure such as a look-up table.

At step 304, in real-time, the current temperature of the retail store in which the product resides is measured. A thermometer may be used to measure the temperature of the store.

At step 306, in real-time, a perishability time for the product is calculated using the average spoilage temperature of the product, and the current temperature of the retail store. In examples, a linear relationship exists between perishability time and the average spoilage temperature of the product. For example, a product when removed from a freezer may increase in temperature by a certain number of degrees per unit time until the product reaches the average spoilage temperature. In other examples, a non-linear relationship between the perishability time and the average spoilage temperature for a product may exist. In many cases, an equation can be used to describe the relationship and to calculate the perishability time.

At step 308, in real-time, the location of the product and the time since the product has left a shelf (or other storage unit) are tracked. The shelf is a location where a shelf temperature is maintained to adequately preserve the product. In other examples, a storage unit (e.g., an open freezer) is maintained at a certain temperature.

In still other examples, the product has an RFID tag or other tracking device. In some examples, tracking the location includes sensing the RFID tag. In some aspects, a RFID reader apparatus transmits signals to RFID tags on the products. The products respond with signals including information. Either the information or the signals themselves can be used by the RFID reader to determine the location of the tag (and hence the product).

In other examples, the tag also measures or senses the temperature of the product. In this way, feedback can be provided to verify the actual temperature of the product.

At step 310, in real-time, the perishability time is compared to the time since the product has left the shelf or storage unit. When the time since the product has left the shelf or storage unit exceeds the perishability time, an action is taken. In some aspects, the action is alerting a customer at a point-of-sale location. In other examples, the action is alerting a customer as the customer traverses through the retail store. In still other examples, the action is alerting an employee of the store. Other examples of actions are possible.

Those skilled in the art will recognize that a wide variety of other modifications, alterations, and combinations can also be made with respect to the above described embodiments without departing from the scope of the invention, and that such modifications, alterations, and combinations are to be viewed as being within the ambit of the inventive concept.

What is claimed is:

1. A system, comprising:
a database configured to store an average spoilage temperature for a product, the average spoilage temperature being the temperature when the product becomes not viable for customer use;
a temperature sensor configured to measure the temperature of the retail store in which the product resides;
a location tracking sensor configured to track the location of the product, and in real-time to track the time since the product left a first shelf, the first shelf being a location where a shelf temperature is maintained to adequately preserve the product;
a control circuit coupled to the database, the temperature sensor, and the location tracking sensor, the control circuit configured to:
in real-time calculate a perishability time for the product using the average spoilage temperature of the product and the current temperature of the retail store;
compare the perishability time to the time since the product has left the first shelf; and
form and transmit a control signal to cause an action to occur when the time since the product has left the first shelf exceeds the perishability time.

2. The system of claim 1, wherein the control signal is an alert to a customer at a point-of-sale location in the retail store.

3. The system of claim 1, wherein the control signal is an alert to a customer as the customer traverses through the retail store.

4. The system of claim 1, wherein the product is coupled to an RFID tag.

5. The system of claim 4, wherein the location sensor senses the RFID tag.

6. The system of claim 1, wherein the control signal is an alert to an employee of the store.

7. The system of claim 1, wherein the average spoilage temperature is a temperature above which the item perishes.

8. The system of claim 1, wherein the average spoilage temperature is a temperature below which the product perishes.

9. A method, comprising:
obtaining an average spoilage temperature for a product, the average spoilage temperature being the temperature when the product becomes not viable for customer use;
in real-time, measuring the current temperature of the retail store in which the product resides;
in real-time, calculating a perishability time for the product using the average spoilage temperature of the product, and the current temperature of the retail store;
in real-time, tracking the location of the product and the time since the product has left a first shelf, the first shelf being a location where a shelf temperature is maintained to adequately preserve the product;
in real-time, comparing the perishability time to the time since the product has left the first shelf; and
when the time since the product has left the first shelf exceeds the perishability time, taking an action.

10. The method of claim 9, wherein the action comprises alerting a customer at a point-of-sale location.

11. The method of claim 9, wherein the action comprises alerting a customer as the customer traverses through the retail store.

12. The method of claim 9, wherein the product has an RFID tag.

13. The method of claim 12, wherein tracking the location comprises sensing the RFID tag.

14. The method of claim 9, wherein the action comprises alerting an employee of the store.

15. The method of claim 9, wherein the average spoilage temperature is a temperature above which the item spoils.

16. The method of claim 9, wherein the average spoilage temperature is a temperature below which the product spoils.
